(19) United States
(12) Patent Application Publication
(10) Pub. No.: US 2017/0365106 A1
(43) Pub. Date: Dec. 21, 2017

(54) METHOD AND APPARATUS FOR AUTOMATIC TRANSMISSION OF MEDICAL DATA

(71) Applicant: FORD GLOBAL TECHNOLOGIES, LLC, Dearborn, MI (US)

(72) Inventors: Oliver LEI, Windsor (CA); Allen R. MURRAY, Lake Orion, MI (US); Tuan Anh BE, Livonia, MI (US)

(21) Appl. No.: 15/184,312

(22) Filed: Jun. 16, 2016

(51) Int. Cl. G07C 5/00; H04W 4/22; G08B 25/10; A61B 5/00; G07C 5/02; G07C 5/08; H04M 3/51; H04W 84/12

(52) U.S. Cl. CPC G07C 5/008; G07C 5/08; H04W 4/22; H04M 3/5116; G08B 25/10; A61B 5/0013; G07C 5/02; A61B 5/747; A61B 5/6893; A61B 5/0077; H04W 84/12

(57) ABSTRACT

A system includes a processor configured to wirelessly receive crash indicia from a vehicle. The processor is also configured to access an occupant profile including medical data relating to an occupant of the vehicle, identify a public safety access point (PSAP), and send the medical data to the identified PSAP, in response to the crash indicia.

### TECHNICAL FIELD

The illustrative embodiments generally relate to a method and apparatus for automatic transmission of medical data.

### BACKGROUND

Numerous advancements have been made in the field of automotive safety with respect to providing assistance to a vehicle involved in an accident. Vehicle sensors are capable of detecting accident occurrence and automatically dialing emergency operators or other parties through vehicle telematics systems. Damaged vehicle systems can self-report or the damage can be detected by on-vehicle sensors. This damage can also be reported to an emergency operator if it is relevant or may affect the type of help provided.

Vehicle cameras can relay images of the interior and exterior environment to emergency operators. Microphones can relay audio and enable occupant communication with an emergency operator. One common feature of most of these accident assistance systems is that the emergency operator or other third-party assistant interacts with the vehicle directly in some manner, or at a minimum exchanges data with the vehicle through a remote intermediary.

### SUMMARY

In a first illustrative embodiment, a system includes a processor configured to wirelessly send an instruction to a remote server to transfer occupant medical data to a public safety assistance point (PSAP) in response to detecting a vehicle accident.

In a second illustrative embodiment, a system includes a processor configured to wirelessly receive crash indicia from a vehicle. The processor is also configured to access an occupant profile including medical data relating to an occupant of the vehicle, identify a public safety access point (PSAP) with which the vehicle is likely to communicate, and send the medical data to the identified PSAP in response to the crash indicia.

In a third illustrative embodiment, a computer-implemented method includes sending vehicle occupant medical information to a public safety access point (PSAP) identified as a PSAP for processing accident data in response to receiving wireless notification from a vehicle reporting an accident. The occupant medical information is retrieved from an occupant profile including previously saved medical information for an occupant currently present in a vehicle reporting the accident.

### BRIEF DESCRIPTION OF THE DRAWINGS

FIG. 1 illustrates an illustrative vehicle computing system;

FIG. 2 illustrates an example of a passenger identification process;

FIG. 3 illustrates an example of an emergency event handling process;

FIG. 4A illustrates an example of profile gathering process;

FIG. 4B illustrates an example of data gathering and delivery process; and

FIG. 5 illustrates an example of a further data gathering process.

### DETAILED DESCRIPTION

As required, detailed embodiments are disclosed herein; however, it is to be understood that the disclosed embodiments are merely illustrative and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the claimed subject matter.

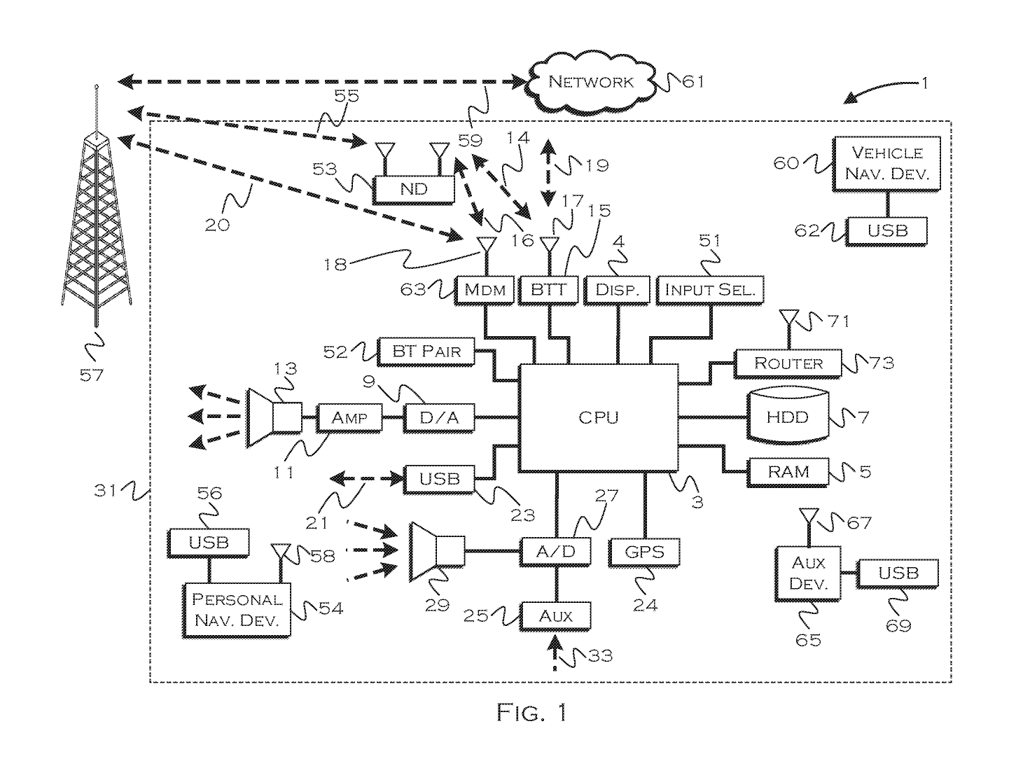

FIG. 1 illustrates an example block topology for a vehicle based computing system 1 (VCS) for a vehicle 31. An example of such a vehicle-based computing system 1 is the SYNC system manufactured by THE FORD MOTOR COMPANY. A vehicle enabled with a vehicle-based computing system may contain a visual front end interface 4 located in the vehicle. The user may also be able to interact with the interface if it is provided, for example, with a touch sensitive screen. In another illustrative embodiment, the interaction occurs through, button presses, spoken dialog system with automatic speech recognition and speech synthesis.

In the illustrative embodiment 1 shown in FIG. 1, a processor 3 controls at least some portion of the operation of the vehicle-based computing system. Provided within the vehicle, the processor allows onboard processing of commands and routines. Further, the processor is connected to both non-persistent 5 and persistent storage 7. In this illustrative embodiment, the non-persistent storage is random access memory (RAM) and the persistent storage is a hard disk drive (HDD) or flash memory. In general, persistent (non-transitory) memory can include all forms of memory that maintain data when a computer or other device is powered down. These include, but are not limited to, HDDs, CDs, DVDs, magnetic tapes, solid state drives, portable USB drives and any other suitable form of persistent memory.

The processor is also provided with a number of different inputs allowing the user to interface with the processor. In this illustrative embodiment, a microphone 29, an auxiliary input 25 (for input 33), a USB input 23, a GPS input 24, screen 4, which may be a touchscreen display, and a BLUETOOTH input 15 are all provided. An input selector 51 is also provided, to allow a user to swap between various inputs. Input to both the microphone and the auxiliary connector is converted from analog to digital by a converter 27 before being passed to the processor. Although not shown, numerous of the vehicle components and auxiliary components in communication with the VCS may use a vehicle network (such as, but not limited to, a CAN bus) to pass data to and from the VCS (or components thereof).

Outputs to the system can include, but are not limited to, a visual display 4 and a speaker 13 or stereo system output. The speaker is connected to an amplifier 11 and receives its signal from the processor 3 through a digital-to-analog converter 9. Output can also be made to a remote BLUETOOTH device such as PND 54 or a USB device such as vehicle navigation device 60 along the bi-directional data streams shown at 19 and 21 respectively.

In one illustrative embodiment, the system 1 uses the BLUETOOTH transceiver 15 to communicate 17 with a user's nomadic device 53 (e.g., cell phone, smart phone, PDA, or any other device having wireless remote network connectivity). The nomadic device can then be used to communicate 59 with a network 61 outside the vehicle 31 through, for example, communication 55 with a cellular tower 57. In some embodiments, tower 57 may be a Wi-Fi access point.

Exemplary communication between the nomadic device and the BLUETOOTH transceiver is represented by signal 14.

Pairing a nomadic device 53 and the BLUETOOTH transceiver 15 can be instructed through a button 52 or similar input. Accordingly, the CPU is instructed that the onboard BLUETOOTH transceiver will be paired with a BLUETOOTH transceiver in a nomadic device.

Data may be communicated between CPU 3 and network 61 utilizing, for example, a data-plan, data over voice, or DTMF tones associated with nomadic device 53. Alternatively, it may be desirable to include an onboard modem 63 having antenna 18 in order to communicate 16 data between CPU 3 and network 61 over the voice band. The nomadic device 53 can then be used to communicate 59 with a network 61 outside the vehicle 31 through, for example, communication 55 with a cellular tower 57. In some embodiments, the modem 63 may establish communication 20 with the tower 57 for communicating with network 61. As a non-limiting example, modem 63 may be a USB cellular modem and communication 20 may be cellular communication.

In one illustrative embodiment, the processor is provided with an operating system including an API to communicate with modem application software. The modem application software may access an embedded module or firmware on the BLUETOOTH transceiver to complete wireless communication with a remote BLUETOOTH transceiver (such as that found in a nomadic device). Bluetooth is a subset of the IEEE 802 PAN (personal area network) protocols. IEEE 802 LAN (local area network) protocols include Wi-Fi and have considerable cross-functionality with IEEE 802 PAN. Both are suitable for wireless communication within a vehicle. Another communication means that can be used in this realm is free-space optical communication (such as IrDA) and non-standardized consumer IR protocols.

In another embodiment, nomadic device 53 includes a modem for voice band or broadband data communication. In the data-over-voice embodiment, a technique known as frequency division multiplexing may be implemented when the owner of the nomadic device can talk over the device while data is being transferred. At other times, when the owner is not using the device, the data transfer can use the whole bandwidth (300 Hz to 3.4 kHz in one example). While frequency division multiplexing may be common for analog cellular communication between the vehicle and the internet, and is still used, it has been largely replaced by hybrids of Code Domain Multiple Access (CDMA), Time Domain Multiple Access (TDMA), Space-Domain Multiple Access (SDMA) for digital cellular communication. If the user has a data-plan associated with the nomadic device, it is possible that the data-plan allows for broad-band transmission and the system could use a much wider bandwidth (speeding up data transfer). In still another embodiment, nomadic device 53 is replaced with a cellular communication device (not shown) that is installed to vehicle 31. In yet another embodiment, the ND 53 may be a wireless local area network (LAN) device capable of communication over, for example (and without limitation), an 802.11g network (i.e., Wi-Fi) or a WiMax network.

In one embodiment, incoming data can be passed through the nomadic device via a data-over-voice or data-plan, through the onboard BLUETOOTH transceiver and into the vehicle's internal processor 3. In the case of certain temporary data, for example, the data can be stored on the HDD or other storage media 7 until such time as the data is no longer needed.

Additional sources that may interface with the vehicle include a personal navigation device 54, having, for example, a USB connection 56 and/or an antenna 58, a vehicle navigation device 60 having a USB 62 or other connection, an onboard GPS device 24, or remote navigation system (not shown) having connectivity to network 61. USB is one of a class of serial networking protocols. IEEE 1394 (FireWire™ (Apple), i.LINK™ (Sony), and Lynx™ (Texas Instruments)), EIA (Electronics Industry Association) serial protocols, IEEE 1284 (Centronics Port), S/PDIF (Sony/Philips Digital Interconnect Format) and USB-IF (USB Implementers Forum) form the backbone of the device-device serial standards. Most of the protocols can be implemented for either electrical or optical communication.

Further, the CPU could be in communication with a variety of other auxiliary devices 65. These devices can be connected through a wireless 67 or wired 69 connection. Auxiliary device 65 may include, but are not limited to, personal media players, wireless health devices, portable computers, and the like.

Also, or alternatively, the CPU could be connected to a vehicle based wireless router 73, using for example a Wi-Fi (IEEE 802.11) 71 transceiver. This could allow the CPU to connect to remote networks in range of the local router 73.

In addition to having exemplary processes executed by a vehicle computing system located in a vehicle, in certain embodiments, the exemplary processes may be executed by a computing system in communication with a vehicle computing system. Such a system may include, but is not limited to, a wireless device (e.g., and without limitation, a mobile phone) or a remote computing system (e.g., and without limitation, a server) connected through the wireless device. Collectively, such systems may be referred to as vehicle associated computing systems (VACS). In certain embodiments particular components of the VACS may perform particular portions of a process depending on the particular implementation of the system. By way of example and not limitation, if a process has a step of sending or receiving information with a paired wireless device, then it is likely that the wireless device is not performing that portion of the process, since the wireless device would not “send and receive” information with itself. One of ordinary skill in the art will understand when it is inappropriate to apply a particular computing system to a given solution.

In each of the illustrative embodiments discussed herein, an exemplary, non-limiting example of a process performable by a computing system is shown. With respect to each process, it is possible for the computing system executing the process to become, for the limited purpose of executing the process, configured as a special purpose processor to perform the process. All processes need not be performed in their entirety, and are understood to be examples of types of processes that may be performed to achieve elements of the invention. Additional steps may be added or removed from the exemplary processes as desired.

With respect to the illustrative embodiments described in the figures showing illustrative process flows, it is noted that a general purpose processor may be temporarily enabled as a special purpose processor for the purpose of executing some or all of the exemplary methods shown by these figures. When executing code providing instructions to perform some or all steps of the method, the processor may be temporarily repurposed as a special purpose processor, until such time as the method is completed. In another example, to the extent appropriate, firmware acting in accordance with a preconfigured processor may cause the processor to act as a special purpose processor provided for the purpose of performing the method or some reasonable variation thereof.

In emergency situations, such as accidents, it is desirable to transfer as much useful information as possible to an emergency operator or first responder. This can include vehicle conditions (fire, rollover, etc.), accident characteristics (front end, speed of collision, side-collision), and location of the vehicle. The more information that emergency operators have or can request, the more precise an emergency response can be. For example, knowing that a vehicle is either on fire or has an increased risk of burning can cause the emergency operator to send a fire department vehicle as well as a police vehicle. Waiting until the police vehicle has arrived to request such services can result in greater injury or damage to occupants and the surrounding environment.

In the proposed illustrative embodiments, a remote server sends advanced information relating to vehicle occupants when a crash occurs. A mobile device or vehicle stores this information initially, and the server obtains the information upon startup, when a crash occurs, or at another suitable time. The vehicle notifies the server (or a system working in conjunction with the server) of the crash, and the server can act to send the stored passenger-specific information via landline or internet connection to the emergency operator. While the vehicle or a mobile device could also send this information, if the emergency operator can only communicate with the vehicle directly using a voice connection, the server's ability to send additional information about the vehicle occupants can provide a useful supplement to the voice call.

Some vehicles are provided with the capability to recognize vehicle occupants by both location and specific identity. These recognition systems may include vehicle cameras with vision identification capability, wireless device sensing technology capable of triangulating a device position and assigning a known user as being located at that position and other similar systems. If a vehicle or vehicle system can specifically identify a user, the vehicle can either identify that user to a remote server for user-related information retrieval or can transmit locally stored user information to the remote server. Also, in some examples, user wireless devices store a user information profile, which may include medical history. The vehicle can access this profile and upload the relevant information to the server. All of these systems can serve to identify vehicle occupants and build at least a temporary emergency dataset relating to vehicle occupants. The server can then send some or all of the data in this dataset to an emergency operator if a crash occurs.

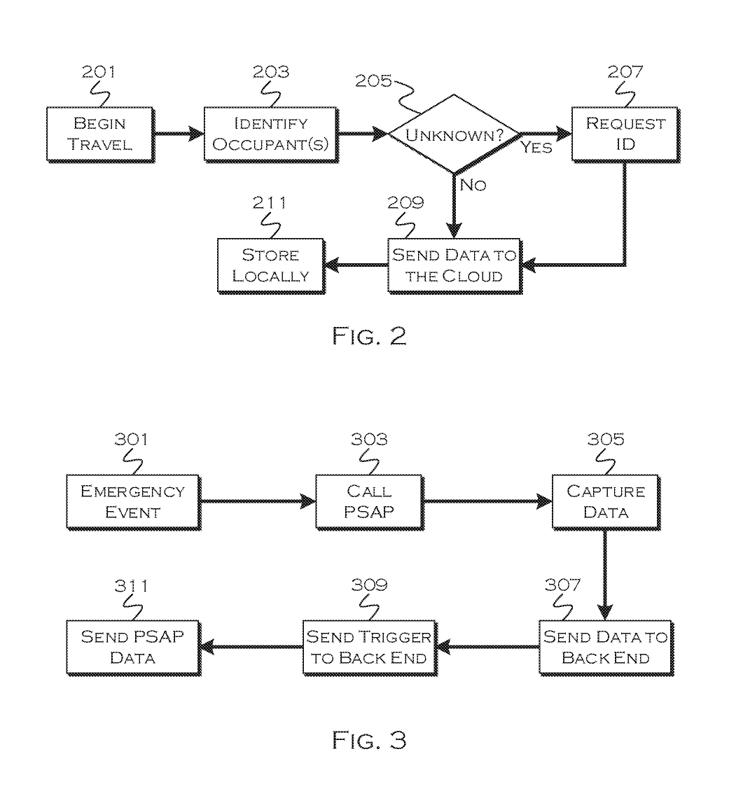

FIG. 2 illustrates an example of a passenger identification process. In this illustrative example, the process will utilize one or more identification subsystems provided to the vehicle for identifying specific passenger locations and/or identities. These systems can include, but are not limited to:
Vision systems—certain cameras can identify both occupant location and identities of previously viewed or known occupants. Identification can be done locally or by a cloud-based system to which the vehicle uploads interior images.
Wireless device identification systems—vehicles may be provided with one or more wireless transceivers that allow the vehicle to locate devices based on signal strength. These systems may also be able to identify specific devices by identification included in device signals. If a device is known to only have a single or common user, identifying a device and/or device location can facilitate user and/or user location identification.
Seat sensors—vehicle weight sensors can identify occupancy or at least the fact that there is significant weight at a seat location. Occupant identity may be determined based on specific occupant weights previously encountered for common occupants This weight-based information may also be coupled with an additional source of information to improve identification accuracy and reliability (e.g., a device has two known users, one weighing approximately 120 lbs and one weighing approximately 200 lbs—if the device is detected at a seat location where approximately 200 lbs is detected, the heavier user may be assumed to be present at that location).
Biometrics—one or more biometric readers may be included in vehicle seats, armrests, ignition systems, etc. Occupant use of a localized biometric reader may identify an occupant for a seat location associated with the localized biometric system.

In the example of FIG. 2, once travel has begun 201, the process identifies as many vehicle occupants as possible 203. The process may be iterative identifying different occupants within the vehicle using different methodologies. The vehicle may use one or more systems and strategies alone or in combination as generally previously described to specifically identify as many occupants as possible. The vehicle can request identification of any occupant 207 that cannot otherwise be identified 205. This can be done through interaction with a human machine interface (HMI) or in any other manner suitable for input of occupant identification.

Once the vehicle has specifically identified or attempted to identify all occupants, the vehicle sends occupant location/identification to a remote server 209. In some examples, one or more occupant devices may store occupant-related medical information, which the vehicle can obtain by requesting the information from the device. In other examples, the vehicle may store this information in conjunction with a previously established user profile. If the vehicle has access to any pertinent medical information, the vehicle may also send this information to the server. In another example, the server may keep the latest medical information of the driver along with the vehicle data previously sent by the driver to the server, possibly using other means such as via the TCU or a web portal, for example. In another example, the vehicle may instruct the device to send occupant/device identification and occupant medical information to the remote server. This strategy avoids storage of sensitive medical information at the vehicle. However, in the example illustrated in FIG. 2, the vehicle locally stores the relevant medical information about some or all occupants 211. As such, the vehicle can use this information on current and future trips with one or more of the same occupants.

FIG. 3 illustrates an example of an emergency event handling process. If a crash or other emergency event occurs 301, the vehicle (or other device executing the process) calls an emergency operator, also known as a public safety answering point (PSAP). This allows for swift initial communication with the PSAP. In some cases, the PSAP can communicate both voice and data with the vehicle, and the vehicle can use locally stored occupant medical data to directly inform the PSAP of occupant medical conditions. Also, in this example, the vehicle cameras and sensors can gather crash-related data 305 when the accident occurs.

In the example illustrated in FIG. 3, the vehicle sends vehicle-related crash data (camera images, sensor data, location data, other data gathered at the point of crash) to the backend cloud server 307. At this time, the vehicle can also transfer occupant medical information to the backend server, if the vehicle has not already sent this information and/or if the backend server cannot otherwise obtain this information (such as from remotely stored profiles). As the data transfer is ongoing, or once the data transfer is complete, the vehicle may also send a trigger signal (or crash indicia) to the backend server to transfer the crash-related data (medical, vehicular, etc.) to the PSAP 309. In response to this signal, the server may transfer the crash-related data to the PSAP. The vehicle can explicitly identify a PSAP based on a signal sent to the backend server, or the backend server may use received vehicle location data or other information to identify a particular PSAP.

It may be unclear at the time of an accident whether a particular PSAP can receive data directly from a vehicle. By sending the data to the remote server, the process achieves at least a redundancy for data transfer. The remote server may be used to send the data because it is faster, because other data is being transferred between the vehicle and PSAP over the voice or other data channel, because the remote server has access to more secondary information, etc. While the illustrative embodiments provide a level of redundancy by sending crash and/or occupant data to the remote server for subsequent relay to the PSAP, there can be a variety of reasons why it is appropriate to use the backend server to transfer crash and occupant related data to the PSAP as well.

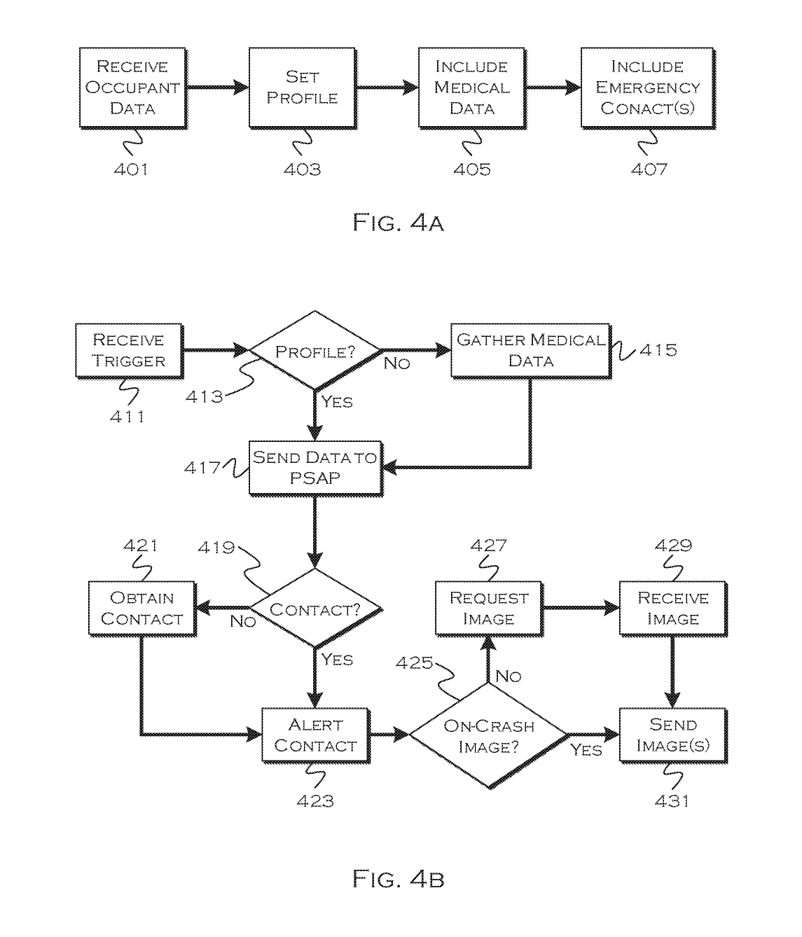

FIG. 4A illustrates an example of a profile gathering process. In this illustrative example, the vehicle builds an occupant medical profile for storage. The vehicle can use this profile at a later time, whenever the vehicle identifies the particular occupant as being present. The vehicle can also upload this information to a backend server and/or delete this information after upload if profile storage is only appropriate at a remote location.

Initially, the vehicle receives occupant identifying data 401. This can include, for example, device identification data, occupant name, occupant weight, occupant biometric data, etc. If this data is repeatedly detectable, the vehicle can detect the same data at a future time and use the data to identify the occupant. It is not necessary to know an occupant name, as the vehicle may use any data that can uniquely identify the occupant or assist in uniquely identifying the occupant.

The vehicle establishes a profile for the occupant based on the identifying data 403. This profile may be stored locally, on a mobile device for later vehicle access and/or in the cloud. Once established, the vehicle also obtains any relevant medical-related data 405 that pertains to the occupant. A user can input this information into the vehicle (using an HMI, for example). In other examples, a mobile device may initially store this information, and the vehicle may obtain the information from the mobile device by request. The vehicle associates the medical information with the profile, for present and future use. In some examples, especially if the information stored is indicative of a non-persistent condition (e.g., pregnancy), the vehicle may only store the information for the duration of a trip, rebuilding the profile as new trips occur. In some examples, the vehicle may store some information persistently (e.g., diabetic) and some information temporarily (e.g., flu).

Also, in this example, the vehicle queries a mobile device (or local in-vehicle contact database) for any emergency contacts associated with the user. These vehicle may also store these contacts with respect to the user profile 407.

FIG. 4B illustrates an example of data gathering and delivery process. In this example, a process executing on the remote server will send relevant emergency data to a PSAP known or expected to be the PSAP contacted by the vehicle. When an accident occurs, the vehicle may send a trigger to the remote server to request transfer of information to the PSAP. In this example, the process begins when the server receives the trigger from the vehicle 411.

Once the trigger is received, the server determines if an occupant profile exists for the particular vehicle 413. This will include, for example, some or all occupants identified as being located in the vehicle, as well as any relevant or obtainable medical information relating to the identified occupants. It is also possible that medical data relating to occupants may not be associated with a specific occupant. For example, the server may have information that one of the occupants is pregnant without a specific occupant identification being established.

If the server and vehicle have not already established a profile for the occupants 413, the process requests medical data from the vehicle 415. While it is often desirable to transfer this data during normal vehicle operation well in advance of any accident, communication issues or other errors may have prevented the data from being previously transferred such that a profile has not yet been established for one or more occupants. Additionally or alternatively, the trigger request itself may be part of a larger data packet containing relevant occupant medical information.

The server sends the relevant medical data obtained to the PSAP 417. The vehicle may explicitly identify the PSAP as part of the trigger request or the server may establish a likely PSAP based on a known vehicle location. Typically, a single PSAP will be assigned to service a particular geographic region such that the server can determine the associated PSAP identity and contact information based on the vehicle location.

In the example of FIG. 4B, the server determines whether there is any emergency contact information associated with one or more of the occupants 419. If the server is not storing any emergency contact information, the server may attempt to obtain this information through a request to the vehicle 421. Once the server has obtained emergency contact information (if any is available), the server will send a communication to the emergency contacts 423. This can include, for example, sending a text message, sending an email, sending a voice message, etc. Since the vehicle is likely engaged in a phone call with the PSAP at this point, the message may also include a reminder that the vehicle occupant may also be so engaged and unavailable to immediately respond to any messages. The message to the emergency contact may also contain other crash-related information as well, which provides an opportunity for a friend or family member to rush to the scene (or a hospital).

The server may also determine whether one or more crash-related camera or sensor images have been received 425. If the server has not received camera/sensor data, the process may request the data from the vehicle 427. The vehicle may initially send this data in response to the trigger request. If the server already has the relevant camera/sensor data stored, or once the server receives the data responsive to the request 429, the process sends the camera and/or sensor images to the PSAP 431.

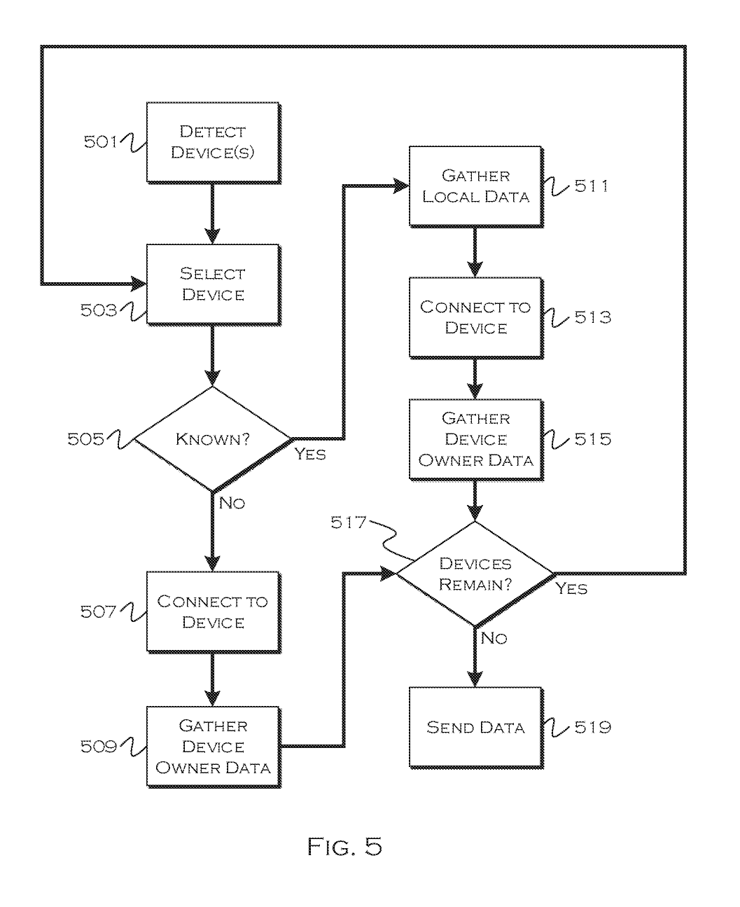

FIG. 5 illustrates an example of a further data gathering process. This illustrative process is a more detailed example of initial data gathering that the vehicle may perform in establishing medical information profiles for detected occupants based on detected devices. In this example, the vehicle detects one or more devices 501. These devices can include, but are not limited to, smartphones, wearables, tablets, medical devices, etc. Many wearables, especially medical devices, may contain some occupant-specific condition information and/or biometric measurements. The vehicle may gather some or all of this data for use in establishing detailed medical information profiles of occupants for use in advanced crash information reporting.

The vehicle selects one of the detected devices 503 and determines if an occupant associated with the device is known 505. If the occupant is known, the vehicle gathers any locally stored data relating to the occupant 511. Since occupant profiles persist locally, in at least one example, the vehicle can access these profiles for use in building medical information profiles for detected occupants. The vehicle may also connect to the device 513 and gather any device-specific data 515 from the device. This can include additional medical information stored on the device, bio-feedback information gathered by the device, emergency contact information stored on the device, physician information stored by the device, etc. The process repeats for each additional device 517 and the vehicle sends the gathered profile and device information to the backend server for use/storage 519.

If the occupant associated with the device is unknown and/or the device is unrecognized, the vehicle attempts to connect to the device 507. If the vehicle is capable of communicating with the device, the vehicle may request specific information from the device. This can include owner identification data, medical data stored on the device, bio-feedback data, physician data, etc. The data gathering process for both known and unknown devices can also include requesting information directly from vehicle occupants through vehicle interfaces. The vehicle can gather occupant identities, medical conditions, and other useful information through user inputs. All of this information can be combined to form a medical response profile for occupants if a crash occurs. The vehicle can subsequently transfer this information to the remote server for use and long and/or short term storage.

Through the use of the illustrative embodiments and similar strategies, additional medical information can be relayed to a PSAP when a crash occurs. By providing a backend server with data transfer capability, the PSAP can obtain relevant information even if direct data communication with the vehicle is not possible.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined in logical manners to produce situationally suitable variations of embodiments described herein.

What is claimed is:

1. A system comprising:
a processor configured to:
wirelessly send an instruction to a remote server to transfer vehicle occupant medical data to a public safety assistance point (PSAP), in response to detecting a vehicle accident.

2. The system of claim 1, wherein the processor is configured to:
determine an identity and location within a vehicle of a vehicle occupant;
obtain medical data relating to an identified vehicle occupant; and
send occupant identification and the obtained related medical data to the remote server.

3. The system of claim 2, wherein the processor is configured to obtain medical data by accessing previously stored medical data relating to the identified vehicle occupant.

4. The system of claim 2, wherein the processor is configured to obtain medical data by requesting medical data from an identified device associated with the vehicle occupant.

5. The system of claim 1, wherein the processor is configured to:
obtain vehicle-related crash data in response to detecting the vehicle accident; and
include the vehicle-related crash data with the instruction, wherein the instruction further comprises an instruction to transfer the vehicle-related crash data to the PSAP.

6. The system of claim 5, wherein the vehicle-related crash data includes a camera image of a vehicle interior.

7. The system of claim 5, wherein the vehicle-related crash data includes vehicle sensor data.

8. A system comprising:
a processor configured to:
wirelessly receive crash indicia from a vehicle;
access an occupant profile including medical data relating to an occupant of the vehicle;
identify a public safety access point (PSAP); and
send the medical data to the identified PSAP in response to the crash indicia.

9. The system of claim 8, wherein the processor is configured to receive the medical data from the vehicle and store the medical data with respect to the occupant profile.

10. The system of claim 8, wherein the processor is configured to receive the medical data included with the crash indicia.

11. The system of claim 8, wherein the processor is configured to receive vehicle-related crash data included with the crash indicia.

12. The system of claim 11, wherein the processor is configured to send the vehicle-related crash data to the identified PSAP.

13. The system of claim 12, wherein the vehicle-related crash data includes vehicle camera images.

14. The system of claim 12, wherein the vehicle-related crash data includes vehicle sensor readings.

15. The system of claim 8, wherein the processor is configured to identify the PSAP based on a vehicle location included with the crash indicia.

16. The system of claim 8, wherein the processor is configured to identify the PSAP based on a PSAP identification included with the crash indicia.

17. A computer-implemented method comprising:
in response to receiving wireless notification from a vehicle reporting an accident, sending vehicle occupant medical information to a public safety access point (PSAP), the occupant medical information retrieved from an occupant profile including previously saved medical information for an occupant currently present in the vehicle reporting the accident.

18. The method of claim 17, further including sending vehicle system information to the PSAP, the system information having been received as part of the wireless notification.

19. The method of claim 17, further including sending an accident notification to an emergency contact retrieved from the occupant profile in response to receiving the wireless notification.

20. The method of claim 17, wherein the PSAP is identified based on vehicle location information included in the notification.
